(19) United States
(12) Patent Application Publication
(10) Pub. No.: US 2026/0192031 A1
(43) Pub. Date: Jul. 9, 2026

(54) METHOD OF INCREASING FLOW RATES IN PNEUMATIC BASED APD SYSTEMS WITHOUT RISK OF PUSHING AIR INTO THE PATIENT

(71) Applicant: GAMBRO LUNDIA AB, Lund (SE)

(72) Inventors: Anbu Aras Arunachalam, Shivamogga (IN); Akhilesh Vibhute, Bangalore (IN); Sachin Sunkad, Bangalore (IN); Belur Shanthakumar Jain, Banglaore (IN); Vinayaka Mutt, Bangalore (IN)

(21) Appl. No.: 19/442,522

(22) Filed: Jan. 7, 2026

(30) Foreign Application Priority Data
Jan. 8, 2025 (IN) 202541001767

(51) Int. Cl. A61M 1/28; A61M 1/14; A61M 1/36

(52) U.S. Cl. CPC A61M 1/282; A61M 1/153; A61M 1/154; A61M 1/155; A61M 1/159; A61M 1/3626; A61M 2205/18; A61M 2205/3334; A61M 2205/3375; A61M 2205/8237

(57) ABSTRACT

An example automated peritoneal dialysis (“APD”) system may include a disposable cassette in fluid communication with a patient line for delivering a dialysis fluid to and from a patient and a cycler comprising at least one pneumatic dialysis fluid pump configured to interact with the disposable cassette to pump the dialysis fluid to and from the patient. The system may further include a sensor located along the patient line configured to output one or more readings indicative of air bubbles in the patient line and a control unit programmed to operate the at least one pneumatic dialysis fluid pump to deliver the dialysis fluid to the patient at a flow rate between 350 ml/min to 450 ml/min. The control unit may be further configured to receive the one or more readings from the sensor and determine an amount of air in the patient line.

### CROSS-REFERENCE TO RELATED APPLICATIONS

The present application claims the benefit of Indian Provisional Application No. 202541001767, entitled METHOD OF INCREASING FLOW RATES IN PNEUMATIC BASED APD SYSTEMS WITHOUT RISK OF PUSHING AIR INTO THE PATIENT and filed Jan. 8, 2025, the contents of which are hereby incorporated by reference in their entirety.

### BACKGROUND

The present invention generally relates to dialysis systems. More specifically, the present invention relates to automated peritoneal dialysis systems. The present invention also relates to methods of performing automated peritoneal dialysis and devices for performing same.

Due to disease, injury, or other causes, a person's renal system can fail. In renal failure of any cause, there are several physiological derangements. The balance of water, minerals, and the excretion of daily metabolic load is no longer possible in renal failure. During renal failure, toxic end products of nitrogen metabolism (urea, creatinine, uric acid, and others) can accumulate in blood and tissues.

Kidney failure and reduced kidney function have been treated with dialysis. Dialysis removes waste, toxins, and excess water from the body that would otherwise have been removed by normal functioning kidneys. Dialysis treatment for replacement of kidney functions is critical to many people because the treatment is life saving. One who has failed kidneys could not continue to live without replacing at least the filtration functions of the kidneys.

Hemodialysis and peritoneal dialysis are two types of dialysis therapies commonly used to treat loss of kidney function. Hemodialysis treatment utilizes the patient's blood to remove waste, toxins and excess water from the patient. The patient is connected to a hemodialysis machine and the patient's blood is pumped through the machine. Catheters are inserted into the patient's veins and arteries to connect the blood flow to and from the hemodialysis machine. As blood passes through a dialyzer in the hemodialysis machine, the dialyzer removes the waste, toxins and excess water from the patient's blood and returns the blood back to the patient. A large amount of dialysate, for example about 120 liters, is used to dialyze the blood during a single hemodialysis therapy. The spent dialysate is then discarded. Hemodialysis treatment lasts several hours and is generally performed in a treatment center about three or four times per week.

Peritoneal dialysis utilizes a dialysis solution or “dialysate”, which is infused into a patient's peritoneal cavity through a catheter implanted in the cavity. The dialysate contacts the patient's peritoneal membrane in the peritoneal cavity. Waste, toxins, and excess water pass from the patient's bloodstream through the peritoneal membrane and into the dialysate. The transfer of waste, toxins and water from the bloodstream into the dialysate occurs due to diffusion and osmosis, i.e., an osmotic gradient occurs across the membrane. The spent dialysate drains from the patient's peritoneal cavity and removes the waste, toxins, and excess water from the patient. This cycle is repeated.

There are various types of peritoneal dialysis therapies, including continuous ambulatory peritoneal dialysis (“CAPD”), automated peritoneal dialysis and continuous flow peritoneal dialysis. CAPD is a manual dialysis treatment, in which the patient connects an implanted catheter to a drain and allows a spent dialysate fluid to drain from the peritoneal cavity. The patient then connects the catheter to a bag of fresh dialysate and manually infuses fresh dialysate through the catheter and into the patient's peritoneal cavity. The patient disconnects the catheter from the fresh dialysate bag and allows the dialysate to dwell within the cavity to transfer waste, toxins and excess water from the patient's bloodstream to the dialysate solution. After a dwell period, the patient repeats the manual dialysis procedure.

In CAPD, the patient performs several drain, fill, and dwell cycles during the day, for example, about four times per day. Each treatment cycle typically takes about an hour. Manual peritoneal dialysis performed by the patient requires a significant amount of time and effort from the patient. This inconvenient procedure leaves ample room for improvement and therapy enhancements to improve patient quality of life.

Automated peritoneal dialysis (“APD”) is similar to CAPD in that the dialysis treatment includes a drain, fill, and dwell cycle. APD machines, however, automatically perform three to four cycles of peritoneal dialysis treatment, typically overnight while the patient sleeps. The APD machines fluidly connect to an implanted catheter. The APD machines also fluidly connect to a source or bag of fresh dialysate and to a fluid drain.

The APD machines pump fresh dialysate from the dialysate source, through the catheter, into the patient's peritoneal cavity and allow the dialysate to dwell within the cavity so that the transfer of waste, toxins and excess water from the patient's bloodstream to the dialysate solution can take place. The APD machines then pump spent dialysate from the peritoneal cavity, though the catheter, to the drain. APD machines are typically computer controlled so that the dialysis treatment occurs automatically when the patient is connected to the dialysis machine, for example, when the patient sleeps. That is, the APD systems automatically and sequentially pump fluid into the peritoneal cavity, allow for a dwell, pump fluid out of the peritoneal cavity and repeat the procedure.

As with the manual process, several drain, fill, and dwell cycles will occur during an APD treatment. A “last fill” is typically used at the end of the APD treatment, which causes dialysis fluid to remain in the peritoneal cavity of the patient when the patient disconnects from the dialysis machine for the day. APD frees the patient from having to manually perform the drain, dwell, and fill steps.

Continuing needs exist to provide improved APD systems. For example, needs exist to provide APD systems with quicker treatment time, such as by increasing the flow rate of current systems. However, increasing the flow rate of APD systems requires an increase in pump pressure, which may introduce other risks to the patient. Accordingly, there is a need for an APD cycler with an increased flow rate while minimizing risks associated with an increased pumping pressure.

### SUMMARY

As discussed above, automated Peritoneal Dialysis (“APD”) cyclers perform sequential exchanges during the night making APD a convenient dialysis therapy. Current APD systems operate to fill a patient at a flow rate around 220 ml/min, resulting in a therapy time of about 2 hours. It may be advantageous to increase the flow rate of APD systems during fill and drain to reduce the therapy time. However, increasing the flow rate may require an increase in pump pressure and/or pump rate, which can lead to the introduction of air into the patient.

Current APD systems prevent air infusion into the patient. For example, the fresh dialysate is always discharged through the bottom portions of pump chambers. This allows entrapped air to rise and be removed from the pump chambers, such as through a drain or to a heater bag. However, these systems may only be effective with lower pump pressures, such as a pump pressure of around +/−1.5 psig. Increasing the pump pressure, which thereby increases the flow rate, may increase the risk of air infusion into the dialysis fluid that is provided to a patient. Entrained air entering a patient's peritoneum during PD can cause discomfort.

The present disclosure provides an automated peritoneal dialysis system and method for detecting entrained air, especially small bubbles of air, in a line running to or from a patient, so that corrective action can be taken.

A first aspect of the present disclosure provides an automated peritoneal dialysis system including a disposable cassette in fluid communication with a patient line for delivering a dialysis fluid to and from a patient; a cycler including at least one pneumatic dialysis fluid pump configured to interact with the disposable cassette to pump the dialysis fluid to and from the patient; a sensor located along the patient line configured to output one or more readings indicative of air bubbles in the patient line; and a control unit. The control unit may be programmed to operate the pneumatic dialysis fluid pump to deliver the fluid to the patient at a flow rate between 350 ml/min to 450 ml/min. The control unit may be further configured to receive the one or more readings from the sensor and determine an amount of air in the patient line

According to a second aspect of the present disclosure, the automated peritoneal dialysis system may include a housing for housing the cycler.

According to a third aspect of the present disclosure, the sensor is located on an external surface of the housing.

According to a fourth aspect of the present disclosure, the housing includes a door and wherein the sensor is located behind the door.

According to a fifth aspect of the present disclosure, the sensor is selected from the group consisting of an optical or infrared (IR) sensor, an ultrasound sensor, and a camera.

According to a sixth aspect of the present disclosure, the sensor is an ultrasound sensor.

According to a seventh aspect of the present disclosure, the control unit is configured to determine air bubbles as small as 20 μL based on the one or more readings of the sensor.

According to an eighth aspect of the present disclosure, the sensor includes a retainer that grasps the patient line.

According to a ninth aspect of the present disclosure, the control unit is further configured to determine if the one or more readings from the sensor is above a pre-determined threshold; and stop the at least one pneumatic dialysis fluid pump if the one or more readings is above the pre-determined threshold.

According to a tenth aspect of the present disclosure, the control unit is further configured to determine if the one or more readings from the sensor is above a pre-determined threshold; and transmit an alarm if the one or more readings is above the pre-determined threshold.

According to an eleventh aspect of the present disclosure, the disposable cassette further includes a plurality of pumping chambers and is in fluid communication with a fresh fluid conduit, the fresh fluid conduit in fluid communication with a supply of fresh dialysis fluid, wherein the fresh fluid conduit is oriented above the patient line.

According to a twelfth aspect of the present disclosure, a pressure of the at least one pneumatic dialysis fluid pump during fill is between 4 and 6 psig.

According to a thirteenth aspect of the present disclosure, the control unit is programmed to operate the at least one pneumatic dialysis fluid pump to drain the dialysis fluid from the patient at a flow rate of 250 ml/min to 300 ml/min.

A fourteenth aspect of the present disclosure provides a peritoneal dialysis (“PD”) method for reducing air entry into a patient. The method includes receiving, via a control unit of a PD machine, an instruction to operate a pneumatic pump of the PD machine at a higher flow rate; signaling, via the control unit, to the pneumatic pump to operate at the higher flow rate; receiving, via the control unit, a signal from a sensor indicative of an amount of air in a patient line of the PD machine; determining, via the control unit, the amount of air in the patient line based on the signal; determining, via the control unit, when the amount of air in the patient line is above a pre-determined threshold; and signaling, via the control unit, to the PD machine to take a remedial action when the amount of air is above the pre-determined threshold.

According to a fifteenth aspect of the present disclosure, the higher flow rate is between 350 ml/min and 450 ml/min.

According to a sixteenth aspect of the present disclosure, the remedial action includes signaling, via the control unit, to the pneumatic pump to operate at a lower flow rate.

According to a seventeenth aspect of the present disclosure, the lower flow rate is between 350 ml/min and 450 ml/min.

According to an eighteenth aspect of the present disclosure, the sensor includes an ultrasonic sensor.

According to a nineteenth aspect of the present disclosure, the remedial action includes signaling, via the control unit, to the PD machine to initiate an alarm.

According to a twentieth aspect of the present disclosure, the remedial action comprises signaling, via the control unit, to the pneumatic pump to stop the pump.

In light of the above aspects and present disclosure set forth herein, it is an advantage of the present disclosure to provide an improved pneumatic based APD system with reduced therapy time.

It is another advantage of the present disclosure to provide a pneumatic based APD system that detects entrained air at higher pumping pressure than traditional systems.

It is a further advantage of the present disclosure to provide a pneumatic based APD system that can determine the amount of entrained air and take corrective action.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1 illustrates a perspective view an automated peritoneal dialysis system with the associated disposable liquid delivery set ready for use with the associated cycler.

FIG. 2 illustrates a perspective view of loading the disposable liquid delivery set and attached cassette that are associated with the system shown in FIG. 1.

FIG. 3 illustrates an air sensor on the patient line according to an example of the present disclosure.

FIG. 4 illustrates an air sensor on the patient line according to another example of the present disclosure.

FIG. 5 illustrates an air sensor on the patient line according to yet another example of the present disclosure.

FIG. 6 illustrates an air sensor on the patient line according to another example of the present disclosure.

FIGS. 7A to 7C illustrate perspective views (FIGS. 7A and 7B) and an exploded view (FIG. 7C) of a battery powered air sensor according to an example of the present disclosure.

FIG. 8 is a process flow diagram of one embodiment of reducing air infusion into a patient.

### DETAILED DESCRIPTION

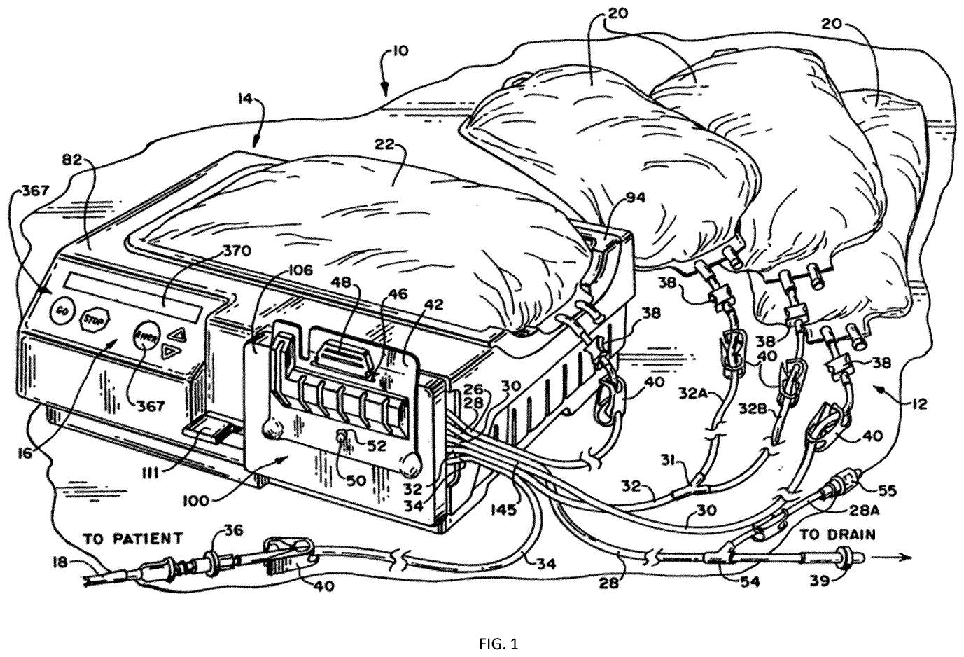

FIG. 1 shows an automated peritoneal dialysis system 10. The system 10 includes three principal components. These are a liquid supply and delivery set 12; a cycler 14 that interacts with the delivery set 12 to pump liquid through it; and a controller 16 that governs the interaction to perform a selected APD procedure. In the illustrated and preferred embodiment, the cycler and controller are located within a common housing 82.

The cycler 14 is intended to be a durable item capable of long term, maintenance free use. The cycler 14 also presents a compact footprint, suited for operation upon a table top or other relatively small surface normally found in the home. The cycler 14 is also lightweight and portable.

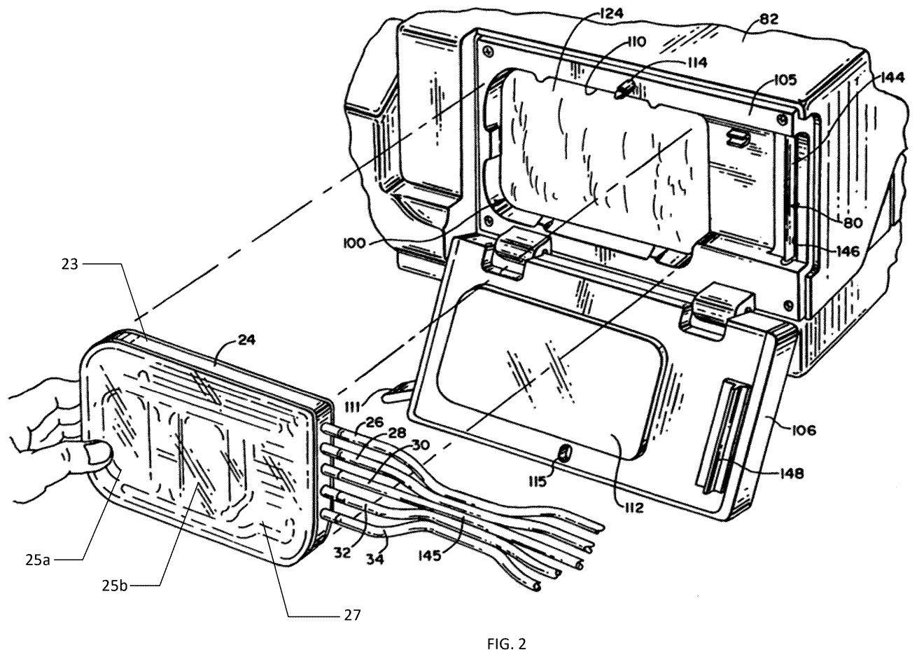

The set 12 is intended to be a single use, disposable item. The user loads the set 12 on the cycler 14 before beginning each APD therapy session. The user removes the set 12 from the cycler 14 upon the completing the therapy session and discards it. As shown in FIG. 2, the set 12 includes a cassette 24 to which lengths of flexible plastic tubes 26, 28, 30, 32, 34 are attached. In use (as FIG. 1 shows), the distal ends of the tubes 26 to 34 connect outside the cycler 14 to the bags 20 of fresh peritoneal dialysis solution, to the liquid heater bag 22, to the patient's indwelling catheter 18, and to a drain (not shown). Liquid heater bag 22 heats the dialysis solution to a desired temperature (typically to about 37 degrees C.) before infusion.

Cassette 24 in one embodiment includes a rigid structure having rigid outer walls 23 and a middle, base wall (not shown) from which pump chambers (25a and 25b as shown in FIG. 2), valve chambers (e.g., volcano valve chambers) and rigid fluid pathways extend. Rigid fluid ports extend from a side wall 23 and communicate fluidly with the rigid cassette pathways and connect sealingly to tubing 26, 28, 30, 32, 34.

Cycler 14 can actuate the pump and valve chambers of cassette 24 pneumatically, mechanically or both. In a preferred embodiment, the pump and valve chambers are pneumatically actuated as shown in the illustrated embodiment. The HomeChoice® APD system uses a pneumatic system described in U.S. Pat. No. 5,350,357 (“the ‘357 Patent”), the entire contents of which are incorporated herein by reference. As seen in FIG. 2, cycler 14 includes a flexible membrane 124, which creates different sealed areas with cassette sheeting 27 at each of the pump and valve chambers of cassette 24. Membrane 124 moves with the sheeting 27 in those areas to either open/close a valve chamber or pump fluid through (into and out of) a pump chamber. An interface plate (not shown) is located behind membrane 124 and includes first and second chamber halves (not shown) that mate with chamber halves 25a and 25b of cassette 24 to form a pair of fixed volume pump chambers.

Cycler 14 in the illustrated embodiment includes a door 106, which closes against cassette 24. Door 106 includes a press plate 112, which can be operated mechanically (e.g., via the closing of the door) and/or pneumatically (e.g., via an inflatable bladder located in the door behind the press plate). Pressing plate 112 against cassette 24 in turn presses cassette 24 against pumping membrane 124, which cooperates with sheeting 27 of cassette 24 to pump fluid through chambers 25a and 25b and to open and close the cassette valve chambers.

The cassette interface plate is located behind membrane 124. Cassette interface plate is configured to apply positive or negative pressure to the cooperating membrane 124 and cassette sheeting 27 at the different valve and pump areas.

For example, positive pressure is applied to membrane 124/sheeting 27 at areas of the membrane/sheeting located within the internal walls of cassette 24 that define pump chambers 25a and 25b to push fluid out of the pump chambers and within the chamber halves of the interface plate (not shown). Negative pressure is applied to membrane 124/sheeting 27 at those same areas to pull fluid into the pump chambers. Likewise, positive pressure is applied to membrane 124/sheeting 27 at areas of the sheeting within the internal walls of cassette 24 and the interface plate defining the valve chambers to close outlet ports of the valve chambers. Negative pressure is applied to membrane 124/sheeting 27 at those same areas to open the outlets of the valve chambers.

The controller 16 paces the cycler 14 through a prescribed series of fill, dwell, drain cycles typical of an APD procedure. The cassette 24 provides centralized valving and pumping functions in carrying out the selected APD therapy. During the fill phase, the cycler 14 infuses the heated dialysate through the set 12 and into the patient's peritoneal cavity. Following the dwell phase, the cycler 14 institutes a drain phase, during which the cycler 14 discharges spent dialysis solution from the patient's peritoneal cavity through the set into a nearby drain (not shown).

In some embodiments, pump chambers 25a and 25b are vertically oriented during use, so air entering the pump chambers 25a, 25b during liquid pumping operations will accumulate near an upper port in each pump chamber 25a, 25b.

The liquid paths and the valve stations are purposefully arranged to isolate the patient's peritoneal cavity from the air that the pump chambers 25a, 25b collect. They are also purposefully arranged so that this collected air can be transferred out of the pump chambers 25a, 25b during use. More particularly, the cassette 24 isolates selected interior liquid paths from the upper ports of the pump chambers 25a, 25b. For example, the patient line 34 that conveys liquid directly to the patient's indwelling catheter can be connected to one of the lower connectors. The cassette 24 thereby isolates these selected liquid paths from the air that accumulates in the pump chambers 25a, 25b. These air-isolated liquid paths (e.g. patient line 34) can be used convey liquid directly into and from the patient's peritoneal cavity.

The cassette 24 also connects other selected liquid paths only to the upper ports of the pump chambers 25a, 25b. These liquid paths can be used to transfer air out of the respective pump chamber 25a, 25b. These liquid paths can also be used to convey liquid away from the patient to other connected elements in the system 10, like the heater bag 22 or the drain. In this way, the cassette 24 serves to discharge entrapped air through established noncritical liquid paths, while isolating the critical liquid paths from the air. However, this air entrapment mechanism may only be effective at lower flow rates and pump pressures below 1.5 psig.

The therapy time is directly influenced by the flow rate of the dialysate through the set 12 (e.g. during fill and/or drain). It may be advantageous for cycler 14 to operate at an increased flow rate to reduce the time required for therapy. In some embodiments, the cycler 14 pumps the heated dialysate into the peritoneal cavity during the fill phase at a flow rate of between 350 ml/min to 450 ml/min, more preferably about 400 ml/min. In some embodiments, the cycler 14 discharges the spent dialysis solution from the patient's peritoneal cavity through the set 12 and into the drain at a flow rate of between 250 ml/min to 300 ml/min, more preferably about 285 ml/min.

Increasing the flow rate of the fluid may require an increase in pumping pressure. In some embodiments, the cycler 14 uses between 4 and 6 psig positive pressure for fill and between 2 and 4 psig negative pressure for drain. An increased flow rate and an increased pumping pressure may increase the risk of air infusion into the patient since prior systems for air entrapment as described above may not be effective at higher pump pressures.

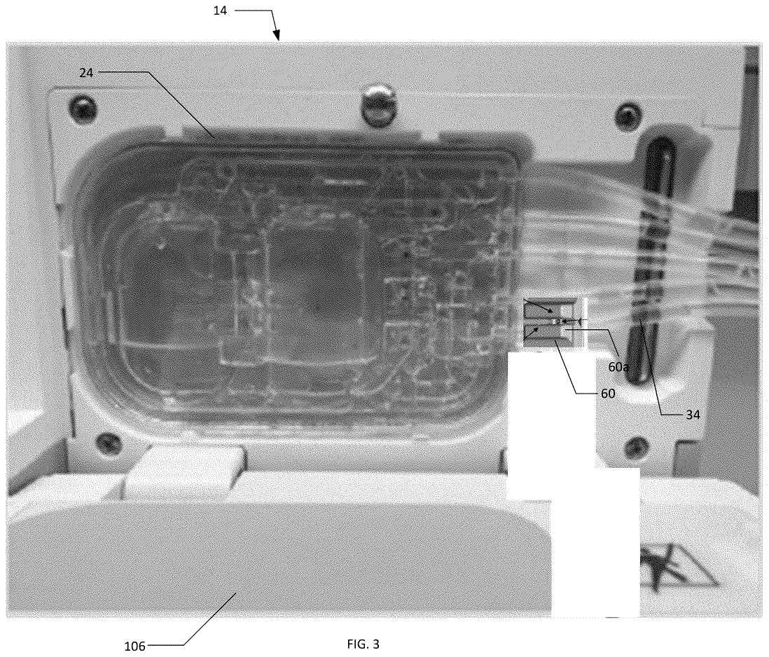

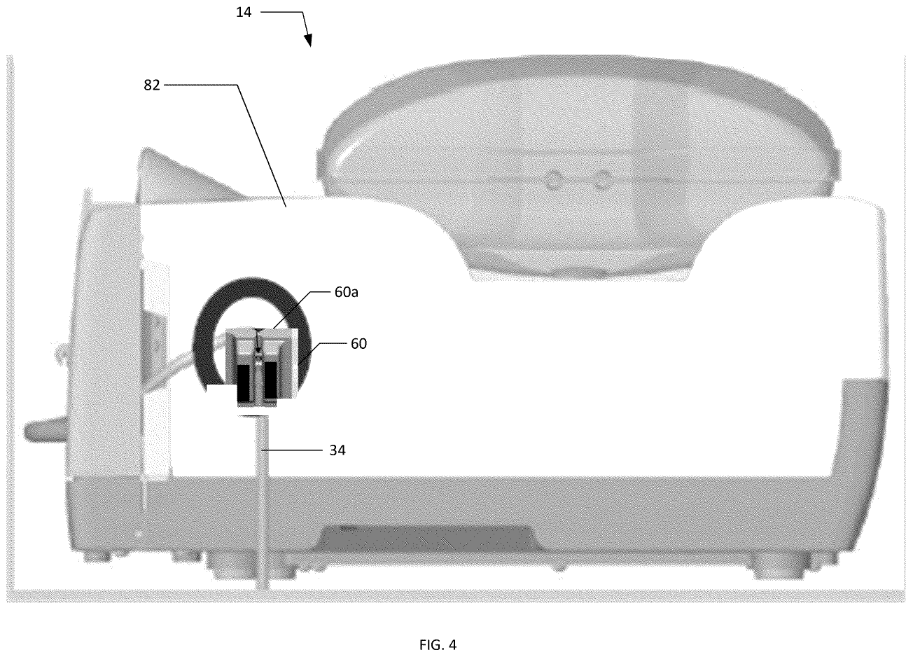

In some embodiments, as shown in FIGS. 3 and 4, the cycler 14 includes a sensor 60 along patient line 34 as a secondary mechanism to detect air. In some embodiments, the sensor 60 is an optical or infrared (IR) sensor, an ultrasound sensor, a camera, or other sensor used to detect air bubbles in fluid know in the art. Sensor 60 includes housing 60a which holds an air detection transmitter and air detection receiver of an air detection sensor in a proper position relative to the patient line 34. Sensor 60 senses any air bubbles that may be entrained in fluid. For example, the sensor 60 may be an inline sensor. As seen, housing 60a includes a clip or retaining ring, that releasably holds patient line 34 in place. Sensor 60 in one embodiment is an ultrasonic sensor, which includes an ultrasonic transmitter and an ultrasonic receiver. Transmitter and receiver can be piezo based transducers, known to those of skill in the art. After a ultrasonic wave has been transmitted, the ultrasonic wave reflects a portion of the wave when encountering an air bubble. This results in a weaker signal at the ultrasonic receiver, which indicates the presence of air.

The sensor 60 may be located on the cycler 14 behind the door 106 of cycler 14 during use as shown in FIG. 3. In some embodiments, when the door 106 is in the closed position, the sensor may interface with the patient line 34. Alternatively, the sensor 60 may be located on an external surface of the cycler 14, such as housing 82 as shown in FIGS. 4 to 6. As shown in the depicted embodiment, the air sensor 60 may be included in a clip where patient line 34 is routed.

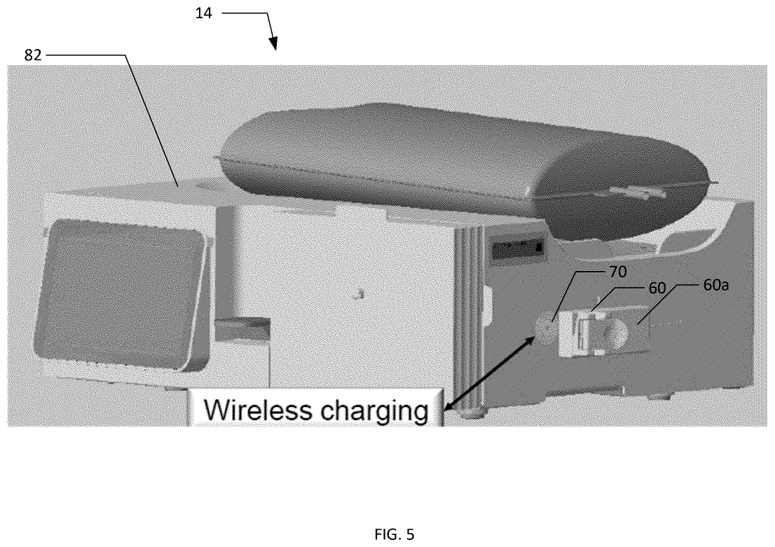

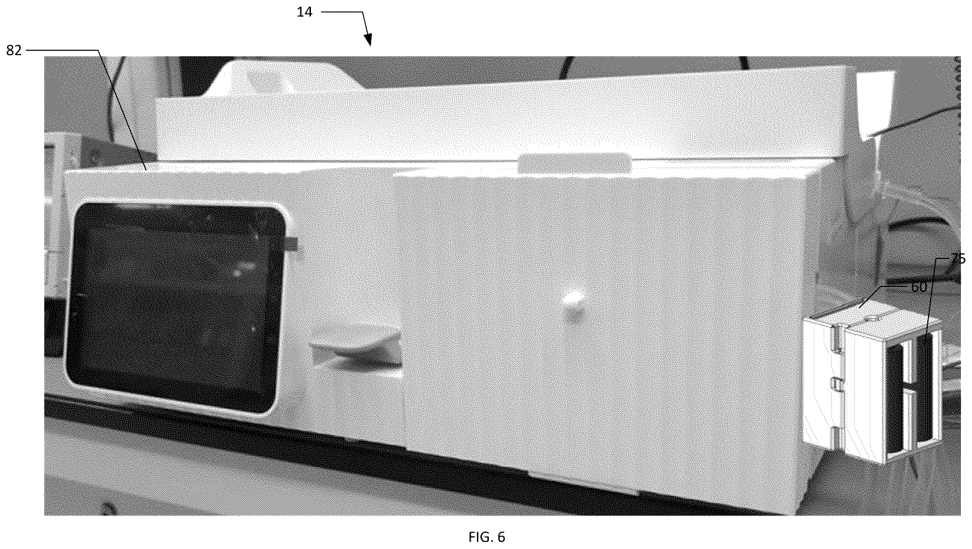

The air sensor 60 may be powered using one more batteries capable of wireless charging as shown in FIG. 5. For example, an external device may be held to a coil 70 on an external surface of the housing 82, which works to charge the battery of the sensor 60 or power the sensor 60 directly. It can be appreciated that other wireless charging techniques may be used. Additionally or alternatively, the air sensor 60 may be powered by one or more conventional batteries 75, such as AA batteries, AAA batteries, or the like, as shown in FIG. 6.

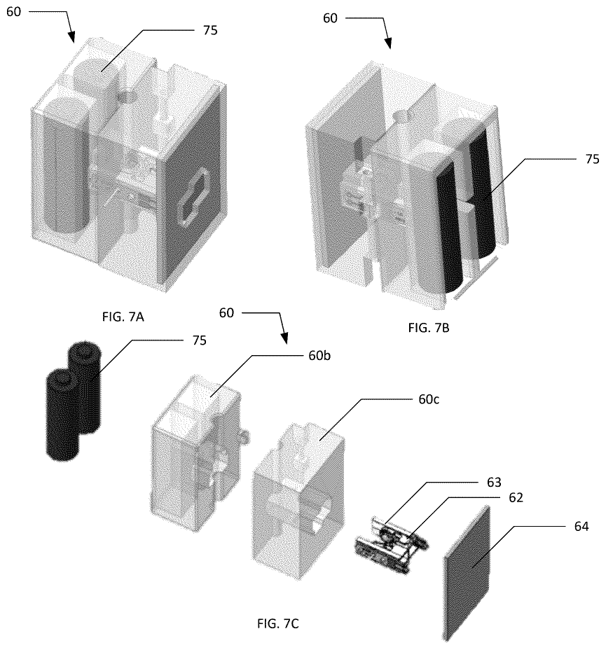

A battery powered air sensor 60 is shown in FIGS. 7A to 7C. The air sensor 60 may include a receiver and a transmitter 62, a clamp 63 for holding the patient line, and one or more batteries 75. The components may be housed within a housing 60b, 60c. As shown in the depicted embodiment, the housing may include a first portion 60b and a second portion 60c. A user may open the first portion 60b relative to the second portion 60c to expose the clamp portion 63 of the sensor 60 for inserting the fluid line of the PD system. The user then may close the first portion 60b of the housing to secure the patient line in place prior to starting treatment.

The battery powered air sensor 60 may be used with existing PD systems. For example, the air sensor 60 may be removably coupled to the housing 82 of the cycler 14. The air sensor may communicate wirelessly with the control unit, such as through via Bluetooth, WiFi, Zigbee, wireless Universal Serial Bus (“USB”), infrared, or any other suitable wireless communication technology. In some embodiments, the air sensor 60 includes a circuit board 64 in operative communication with the air sensor 60 including one or more components for wireless transmission.

While the depicted embodiments illustrate an APD system including a cassette, it can be appreciated that the air sensor 60 disclosed herein and methods for detecting entrained air may be used with other APD systems. For example, in some embodiments, the APD system may include a cycler with reusable fluidics in fluid communication with a patient line and/or drain line provided to a patient catheter (as opposed to the use of a cassette). The air sensor 60 may be positioned upon the reusable patient line of the APD system to detect air within the line before reaching the patient.

In some embodiments, the air sensor 60 can be external to the cycler, such as on an outside surface of the housing. Similar to the APD system with the cassette, the reusable patient line can be placed within the external air sensor 60 prior to treatment. Additionally or alternatively, the air sensor 60 may be integrated into the cycler, such within the housing of the cycler. The air sensor may be located on a portion of the reusable fluid line downstream from a pump, which may be a peristaltic pump used to pump fluid from the cycler to the patient.

The sensor 60 outputs one or more readings indicative of the presence or lack of air in the patient line 34 to control unit 16. For example, air sensor 60 may send the output to the control unit 16, where the control unit 16 may determine an amount of air in the patient line 34. If the air in the patient line is above a pre-determined threshold, the control unit 16 may alarm the user (such as a patient or medical professional) of air in the patient line 34. Additionally or alternatively, the control unit 16 may stop the pump if the output of sensor 60 is indicative of an amount of air in the patient line 34 exceeding a predetermined threshold. In some embodiments, the sensor 60 can detect air bubbles about 20uL or greater.

In some embodiments, when the control unit 16 determines air above a pre-determined threshold, the control unit 16 may automatically initiate an operation to purge the air from the patient line 34. Air purging may include the control unit 16 sending a signal to the pump to reverse the direction of the pump flow to pump a pre-determined amount of fluid back to the pumping chamber. Reversing the direction pulls the fluid containing the air back away from the patient to the pumping chamber. Because the air sensor is in a stationary position relative to the pump chamber, the control unit 16 can calculate the amount of fluid that needs to be pulled back for the air to reach the pumping chamber.

In the pumping chamber, air will settle to the top of the pumping chamber. The control unit 16 may send a signal to the pump to pump the fluid and air in the pumping chamber into the drain line. In some embodiments, a high pressure, such as 5 to 7 psig will be applied to the pumping chamber to push the entire contents of the pumping chamber, including the air, into the drain. The control unit 16 may then send a signal to the pump to return the flow of fluid to the patient to continue treatment. The air purging operation allows the cycler to remove detected air in the patient line without intervention from the patient or caregiver.

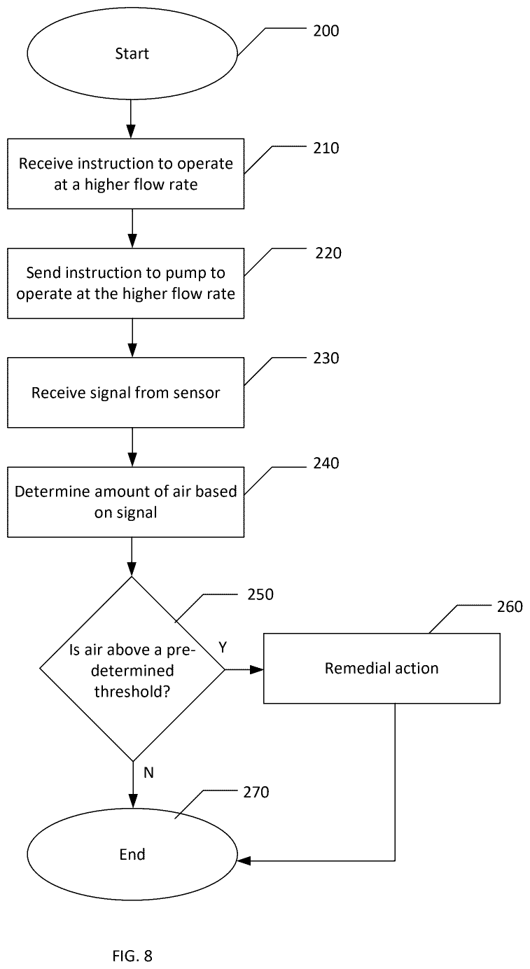

FIG. 8 shows one method which may be implemented in control unit 16 for reducing air infusion into the patient. At block 210 the control unit 16 receives instructions to operate the cycler 14 at a higher flow rate, such as between 350 ml/min to 450 ml/min. In some embodiments, the control unit 16 receives the instruction based on user input at a user interface of cycler 14. At block 220, the control unit 16 sends instructions to the pneumatic pump to operate at the higher flow rate. The cycler 14 will then operate at an increased flow rate to reduce the time required for therapy. As discussed previously, increasing the flow rate, which thereby increases the pump pressure, may prevent the removal of entrapped air using existing techniques in pneumatic pump systems.

During treatment, the sensor 60 will output one or more readings indicative of the presence or lack of air in the patient line 34. At block 230, the control unit 16 receives the signal from sensor 60 and determines the amount of air in the patient line based on the sensed signal at block 240. At diamond 250, the control unit 16 may then determine if the air in the patient line 34 is above or below a predetermined threshold.

At diamond 250, when the air in the patient line 34 is above the pre-determined threshold, method moves to block 260 where a remedial action is taken to reduce air infusion into the patient. In some embodiments, the remedial action may include control unit 16 causing cycler 14 to issue an alarm to the user. In some embodiments, the remedial action may include the control unit 16 sending a signal to stop the pump. In other embodiments, the remedial action may include automatically sending a signal to the pump to decrease the pump speed from the higher flow rate to a lower flow rate, such as between 200 ml/min and 300 ml/min. Reducing the flow rate, which reduces the pumping pressure, may allow the existing air removal methods to become effective. In yet another embodiment, the remedial action may include automatically initiating the air purging operation as discussed above to remove air from the patient line without intervention from the patient or caregiver.

The invention is claimed as follows:

1. An automated peritoneal dialysis system comprising:
a disposable cassette in fluid communication with a patient line for delivering a dialysis fluid to and from a patient;
a cycler comprising at least one pneumatic dialysis fluid pump configured to interact with the disposable cassette to pump the dialysis fluid to and from the patient;
a sensor located along the patient line configured to output one or more readings indicative of air bubbles in the patient line; and
a control unit programmed to operate the at least one pneumatic dialysis fluid pump to deliver the dialysis fluid to the patient at a flow rate between 350 ml/min to 450 ml/min, the control unit further configured to receive the one or more readings from the sensor and determine an amount of air in the patient line.

2. The automated peritoneal dialysis system of claim 1, further comprising a housing for housing the cycler.

3. The automated peritoneal dialysis system of claim 2, wherein the sensor is located on an external surface of the housing.

4. The automated peritoneal dialysis system of claim 2, wherein the housing comprises a door and wherein the sensor is located behind the door.

5. The automated peritoneal dialysis system of claim 1, wherein the sensor is selected from the group consisting of an optical or infrared (IR) sensor, an ultrasound sensor, and a camera.

6. The automated peritoneal dialysis system of claim 1, wherein the sensor is an ultrasound sensor.

7. The automated peritoneal dialysis system of claim 1, wherein the control unit is configured to determine air bubbles as small as 20 μL based on the one or more readings of the sensor.

8. The automated peritoneal dialysis system of claim 1, wherein the sensor comprises a retainer that grasps the patient line.

9. The automated peritoneal dialysis system of claim 1, wherein the control unit is further configured to:
determine when the one or more readings from the sensor is above a pre-determined threshold; and
stop the at least one pneumatic dialysis fluid pump when the one or more readings is above the pre-determined threshold.

10. The automated peritoneal dialysis system of claim 1, wherein the control unit is further configured to:
determine when the one or more readings from the sensor is above a pre-determined threshold; and
transmit an alarm when the one or more readings is above the pre-determined threshold.

11. The automated peritoneal dialysis system of claim 1, wherein the disposable cassette further comprises a plurality of pumping chambers and is in fluid communication with a fresh fluid conduit, the fresh fluid conduit in fluid communication with a supply of fresh dialysis fluid, wherein the fresh fluid conduit is oriented above the patient line.

12. The automated peritoneal dialysis system of claim 1, wherein a pressure of the at least one pneumatic dialysis fluid pump during fill is between 4 and 6 psig.

13. The automated peritoneal dialysis system of claim 1, wherein the control unit is programmed to operate the at least one pneumatic dialysis fluid pump to drain the dialysis fluid from the patient at a flow rate of 250 ml/min to 300 ml/min.

14. A peritoneal dialysis (“PD”) method for reducing air entry into a patient, the method comprising:
receiving, via a control unit of a PD machine, an instruction to operate a pneumatic pump of the PD machine at a higher flow rate;
signaling, via the control unit, to the pneumatic pump to operate at the higher flow rate;
receiving, via the control unit, a signal from a sensor indicative of an amount of air in a patient line of the PD machine;
determining, via the control unit, the amount of air in the patient line based on the signal;
determining, via the control unit, when the amount of air in the patient line is above a pre-determined threshold; and
signaling, via the control unit, to the PD machine to take a remedial action when the amount of air is above the pre-determined threshold.

15. The PD method of claim 14, wherein the higher flow rate is between 350 ml/min and 450 ml/min.

16. The PD method of claim 15, wherein the remedial action comprises: signaling, via the control unit, to the pneumatic pump to operate at a lower flow rate.

17. The PD method of claim 16, wherein the lower flow rate is between 350 ml/min and 450 ml/min.

18. The PD method of claim 14, wherein the sensor comprises an ultrasonic sensor.

19. The PD method of claim 14, wherein the remedial action comprises: signaling, via the control unit, to the PD machine to initiate an alarm.

20. The PD method of claim 14, wherein the remedial action comprises: signaling, via the control unit, to the pneumatic pump to stop the pneumatic pump.
